(19) United States
(12) Patent Application Publication
(10) Pub. No.: US 2023/0320766 A1
(43) Pub. Date: Oct. 12, 2023

(54) MEDICAL INSTRUMENT GRIP HOLDER AND SPINAL SURGICAL INSTRUMENT

(71) Applicant: KYOCERA Corporation, Kyoto-shi, Kyoto (JP)

(72) Inventor: Koichi KATO, Daito-shi, Osaka (JP)

(21) Appl. No.: 18/021,087

(22) PCT Filed: Sep. 1, 2021
(86) PCT No.: PCT/JP2021/032099

§ 371 Date: Feb. 13, 2023

(30) Foreign Application Priority Data
Sep. 2, 2020 (JP) 2020-147851

(51) Int. Cl. A61B 17/70

(52) U.S. Cl. CPC A61B 17/7083

(57) ABSTRACT

A medical instrument grip holder includes an outer shaft, a guide sleeve, and a stopper having a cylindrical shape. The stopper includes, on a proximal end portion, a second flange in contact with a proximal end of a grip. The stopper includes, on an inner peripheral surface of a cylindrical portion of the stopper, a projecting portion configured to engage with a recessed portion disposed on a cylindrical surface of the guide sleeve when the guide sleeve is inserted to a predetermined position. The guide sleeve is housed in an inner portion of the grip with the outer shaft and the stopper assembled from opposite directions along an axis line of a cylinder of the guide sleeve.

### TECHNICAL FIELD

The present disclosure relates to a medical instrument grip holder and a spinal surgical instrument used in spinal surgery.

### BACKGROUND OF INVENTION

Known surgery for fixation of a spine includes surgery in which a plurality of spinal implants are implanted in a spine, and a fixation rod is inserted into a slit of each of the implants so as to extend over the plurality of implants thereby fixing a plurality of vertebrae together. An instrument called a rod inserter accurately inserts the fixation rod into the slit of each of the implants.

A surgeon uses the instrument by gripping a grip of the rod inserter, inserting the rod held at a tip of the rod inserter into a patient body, and disposing the rod at a predetermined position.

A shell positioner used in total hip arthroplasty is also a surgical instrument including a grip. A known grip of the shell positioner holds a shell, the shell being a member constituting an artificial hip joint, and has grip performance and safety.

### SUMMARY

In one aspect of the present disclosure, a medical instrument grip holder includes a first cylindrical member, a junction member, and a stopper having a cylindrical shape. The stopper includes, on a proximal end portion, a flange in contact with a proximal end of a grip held by the medical instrument grip holder. The stopper includes, on an inner peripheral surface of a cylindrical portion of the stopper, a projecting portion configured to engage with a recessed portion disposed on a cylindrical surface of the junction member when the junction member is inserted to a predetermined position. The junction member is housed in an inner portion of the grip with the first cylindrical member and the stopper assembled from opposite directions along an axis line of a cylinder of the junction member.

In one aspect of the present disclosure, a spinal surgical instrument includes the medical instrument grip holder, a grip, a second arm including a proximal end portion configured to connect to the first cylindrical member, and configured to hold a rod on a distal end portion, a second cylindrical member disposed in the cylinder of the first cylindrical member, a first arm including a proximal end portion configured to connect to the second cylindrical member, and configured to attach and detach the rod by moving a distal end portion, and a knob configured to connect to the second cylindrical member, and configured to operate a movement of the distal end portion of the first arm.

### BRIEF DESCRIPTION OF THE DRAWINGS

FIG. 1 is a perspective view illustrating an overview of a rod inserter.

FIG. 2 is an exploded perspective view illustrating a component part of the rod inserter.

FIG. 3 is a schematic view illustrating a mounting position of the component part of the rod inserter.

FIG. 4 is a schematic perspective view illustrating details of a grip holding portion of the rod inserter.

FIG. 5 is a perspective view illustrating an outer appearance of a stopper.

FIG. 6 is a schematic plan view illustrating a configuration of a projecting portion of the stopper.

FIG. 7 is a perspective view illustrating an outer appearance of an outer shaft.

FIG. 8 is a schematic view illustrating a groove provided in the outer shaft.

FIG. 9 is a perspective view illustrating an outer appearance of a guide sleeve.

FIG. 10 is a schematic view illustrating a method for connecting the stopper and the guide sleeve.

FIG. 11 is a schematic view illustrating a method for detaching the stopper from the guide sleeve.

FIG. 12 is a schematic view illustrating a connection state between the outer shaft and the guide sleeve.

FIG. 13 is a schematic view illustrating a method for connecting the outer shaft and the guide sleeve.

FIG. 14 is a schematic view illustrating the method for connecting the outer shaft and the guide sleeve.

### DESCRIPTION OF EMBODIMENTS

A surgical instrument is used in surgery, and is thus preferably washed after surgery in pieces that are as small as possible. The surgical instrument can be preferably disassembled into small pieces.

On the other hand, when the surgical instrument can be easily disassembled, parts and the like may fall into a body during surgery, and thus it is also desirable for the surgical instrument to be less likely to disassemble.

According to the present disclosure, a projecting portion of a stopper and a recessed portion of a junction member (also referred to as a guide sleeve) engage with each other. Thus, the projecting portion and the recessed portion are positioned and fixed in an engagement position. In this way, the stopper and the junction member are fixed to each other, and the junction member and a first cylindrical member (also referred to as an outer shaft) are housed and fixed in an inner portion of a grip, and thus the stopper and the first cylindrical member are fixed at a predetermined position. Accordingly, a medical instrument grip holder that cannot be easily disassembled can be realized. The stopper and the junction member can be disassembled by releasing the engagement between the projecting portion of the stopper and the recessed portion of the junction member. Since the stopper, the junction member, and the first cylindrical member can be disassembled by performing a predetermined procedure, an instrument can be repeatedly disassembled, washed, and then assembled.

A medical instrument grip holder 100 and a rod inserter 1 according to an embodiment of the present disclosure will be described below in detail. For example, when posterior lumbar spinal fusion surgery is performed by using a percutaneous pedicle screw (PPS), the rod inserter 1 according to the present embodiment is used for inserting a rod for fixing an inserted spinal implant. Note that, herein, an example in which the medical instrument grip holder 100 is applied to the rod inserter 1 (a spinal surgical instrument) will be described, but application of the medical instrument grip holder 100 is not limited to the example. The medical instrument grip holder 100 can be applied to various instruments used in medical care including a grip.

Overview of Rod Inserter 1

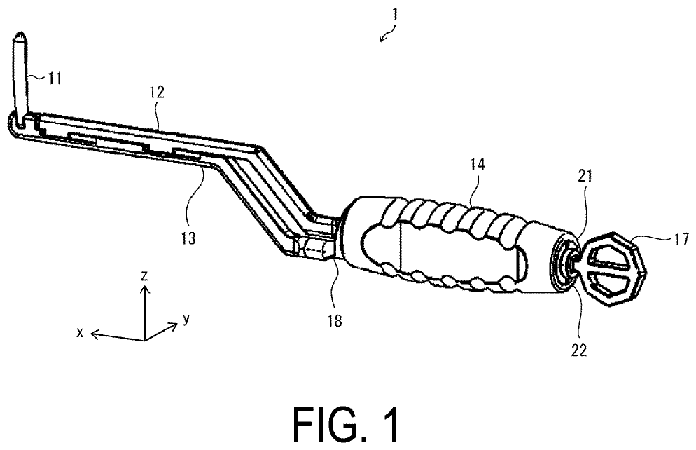

First, an overview of the rod inserter 1 (spinal surgical instrument) will be described with reference to FIG. 1. FIG. 1 is a perspective view of the rod inserter 1 according to the present embodiment. Note that, as illustrated in FIG. 1, in the present embodiment, a direction from a grip 14 to a rod 11 is an x direction, a right direction when the rod 11 is viewed from the grip 14 is a y direction, and a direction in which the rod 11 stands is a z direction. A side closer to an operator who operates the rod inserter 1 is a proximal side, and a side farther from the operator is a distal side.

As illustrated in FIG. 1, the rod inserter 1 includes a first arm 12 and a second arm 13 assembled on one side of the grip 14. The rod 11 is mounted on a distal end of the first arm 12 and the second arm 13 on an opposite side to the grip 14. A knob 17 can be mounted on a proximal end of the grip 14. By rotating the knob 17, the first arm 12 moves in the +x direction or the −x direction, and the rod 11 can be held or released.

The grip 14 is sandwiched between an outer shaft 18 and a stopper 22 as described in detail below. Note that a mechanism for sandwiching the grip 14 between the outer shaft 18 and the stopper 22 is also referred to as the medical instrument grip holder 100.

Internal Structure of Rod Inserter 1

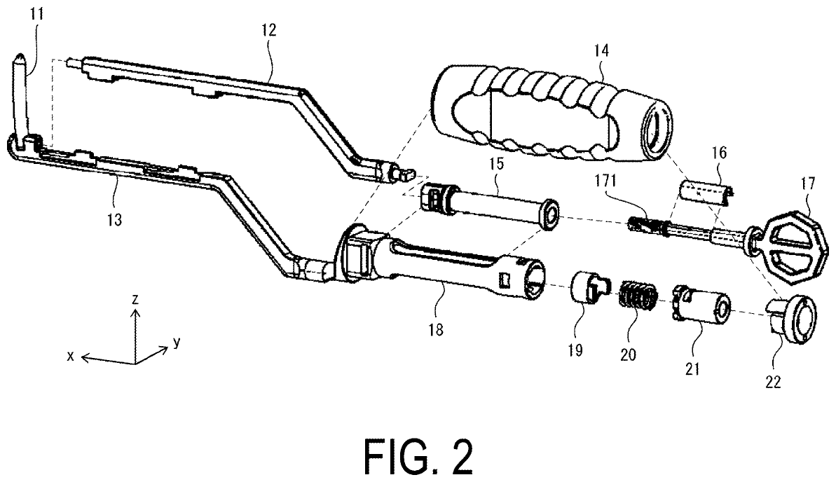

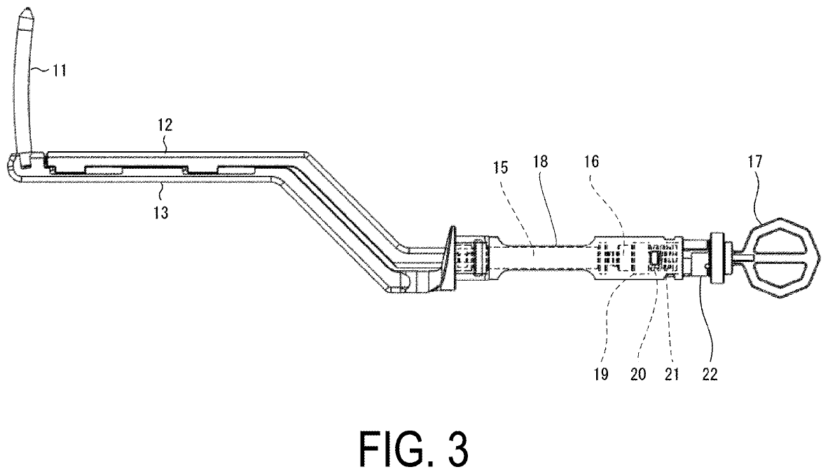

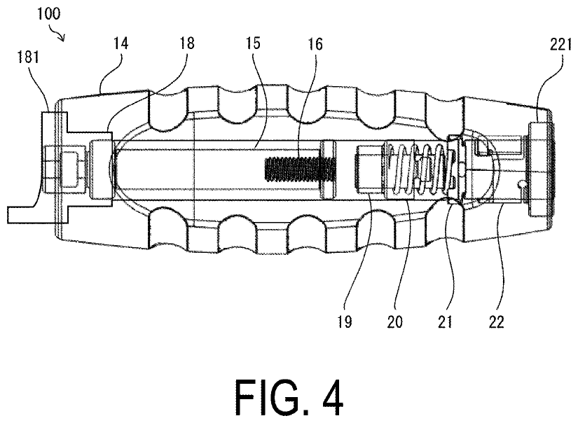

An internal structure of the rod inserter 1 will be described with reference to FIGS. 2 to 4. FIG. 2 is an exploded perspective view illustrating component parts of the rod inserter 1. FIG. 3 is a schematic view illustrating a mounting position of each component part. FIG. 4 is a schematic perspective view illustrating details of the medical instrument grip holder 100 holding the grip 14. Note that FIG. 3 does not explicitly illustrate the grip 14 in order to clarify the mounting position of the component part. FIG. 4 illustrates each part by a solid line in order to clarify the component parts inside the grip 14, but only schematically illustrates each part. Each part inside the grip 14 cannot be confirmed from the outside in the grip 14 that is normal.

As illustrated in FIG. 2, the rod inserter 1 includes the first arm 12, the second arm 13, the grip 14, a shaft (second cylindrical member) 15, a spacer 16, the knob 17, the outer shaft (first cylindrical member) 18, a spring holder 19, a spring 20, a guide sleeve (junction member) 21, and the stopper 22. The spring 20 is an urging member and is a so-called coil spring in the present embodiment, but is not limited to this, and may be formed of a plate spring or an elastic polymer.

The first arm 12 is connected to the shaft 15, and the shaft 15 is connected to the knob 17. A connecting portion between the shaft 15 and the knob 17 is a screw. The knob 17 is rotated to change a distance between the shaft 15 and the knob 17. In this way, the first arm 12 can move in the +x direction or the −x direction. The spacer 16 is mounted on a rod-shaped portion of the knob 17, and the distance between the knob 17 and the shaft 15 does not deviate from a predetermined range. The spacer 16 is disposed in an inner portion of the spring holder 19 and the spring 20.

The rod 11 is placed at the distal end of the second arm 13. The first arm 12 moves in the x direction, and thus the distal end of the first arm 12 enters a recessed portion (not illustrated) of the rod 11 thereby fixing the rod 11. Conversely, the first arm 12 moves in the −x direction, and thus the distal end of the first arm 12 leaves the rod 11 thereby detaching the rod 11 from the rod inserter 1. The mechanism can attach and detach the rod inserter 1 and the rod 11.

The second arm 13 is connected to the outer shaft 18. As illustrated in FIG. 3, the shaft 15 is assembled into the outer shaft 18, and the spring holder 19, the spring 20, the guide sleeve 21, and the stopper 22 are also assembled in an order from the second arm 13 side.

As illustrated in FIG. 4, the grip 14 is sandwiched between a first flange 181 of the outer shaft 18 and a second flange 221 of the stopper 22. In other words, the outer shaft 18, the spring holder 19, the spring 20, the guide sleeve 21, and the stopper 22 constitute the medical instrument grip holder 100. In other words, the medical instrument grip holder 100 includes the outer shaft 18, the guide sleeve 21, and the stopper 22 having a cylindrical shape.

Details of a method for assembling each part will be described below. In the medical instrument grip holder 100, the stopper 22 and the guide sleeve 21 are assembled, and the guide sleeve 21 and the outer shaft 18 are also assembled to assemble the stopper 22 and the outer shaft 18 together. With the grip 14 sandwiched between the second flange 221 of the stopper 22 and the first flange 181 of the outer shaft 18, the medical instrument grip holder 100 that does not easily disassemble can be realized.

An inner portion of the outer shaft 18 houses the shaft 15 and also a part of the guide sleeve 21. The inner portion of the outer shaft 18 houses the spring holder 19 and the spring 20, and the spring 20 urges the guide sleeve 21 in an opposite direction to the first flange 181 of the outer shaft 18.

The guide sleeve 21 has a hole in an axial direction into which a shaft portion of the knob 17 is inserted. A screw hole portion 151 of the shaft 15 and a screw portion 171 provided in a distal portion of the shaft portion of the knob 17 also mesh with each other as screws. In other words, the shaft portion of the knob 17 is connected to the shaft 15 through an inner portion of the guide sleeve 21 and through the inner portion of the spring 20 and the spring holder 19. The knob 17 is rotated to change the distance between the knob 17 and the shaft 15, and thus the first arm 12 connected to the shaft 15 moves in the axial direction.

The spring 20 is held by the spring holder 19 inside a cylindrical portion 182 of the outer shaft 18, and urges the guide sleeve 21 toward the stopper 22. As described below, the guide sleeve 21 is urged toward the stopper 22 to make protruding portions 211 of the guide sleeve 21 less likely to detach from a third groove portion 185 of the outer shaft 18, and the outer shaft 18 and the guide sleeve 21 can be made less likely to detach.

Details of Component Parts

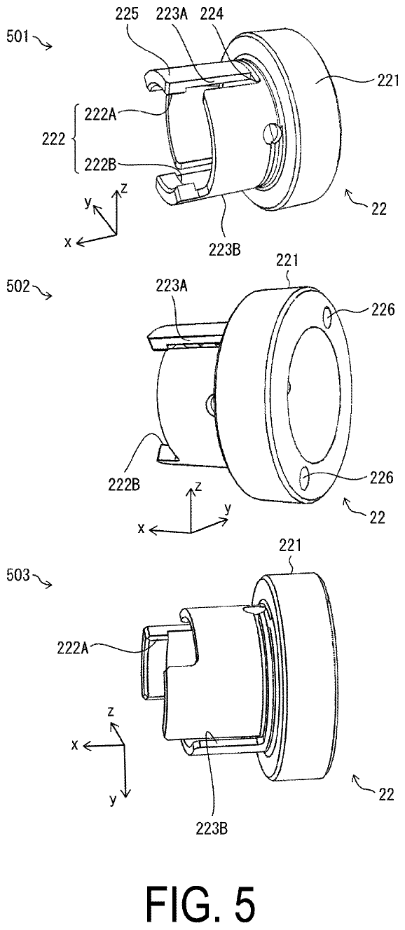

Details of each component part will be described with reference to FIGS. 5 to 9.

Stopper 22

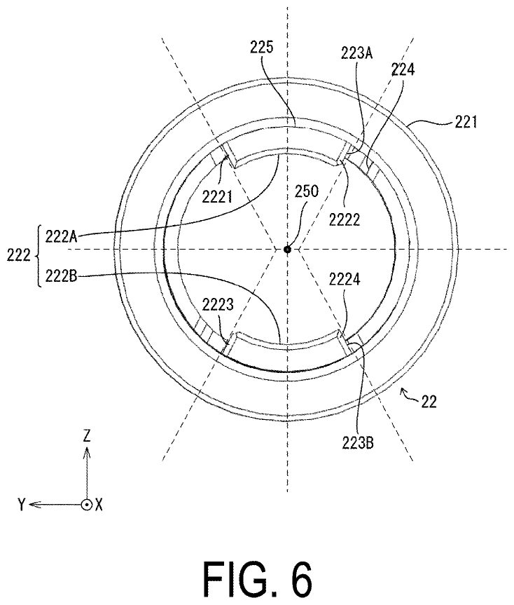

First, the stopper 22 will be described with reference to FIGS. 5 and 6. FIG. 5 is a perspective view illustrating details of the stopper 22. FIG. 6 is a schematic plan view illustrating projecting portions 222 of the stopper 22. As illustrated in the diagram with a reference sign 501 in FIG. 5, the stopper 22 has a cylindrical shape, and includes the second flange 221 at a proximal end portion of a cylindrical portion 225. The cylindrical portion 225 is provided with slits 223 (223A and 223B) from an opposite side to the second flange 221. The slits 223 are bent in a circumferential direction in the cylindrical portion 225 near the second flange 221.

The projecting portions 222 (222A and 222B) protruding toward an inner peripheral side of the cylinder are provided at a distal end portion of the cylindrical portion 225 on an opposite side to the second flange 221. As illustrated in the diagram with a reference sign 502, a wrench hole 226 is provided in a surface of the second flange 221 on an opposite side to the cylindrical portion 225. Insertion of a wrench into the wrench hole 226 can facilitate rotation of the stopper 22 in the circumferential direction.

Note that, in the present embodiment, two projecting portions 222 are provided at positions facing each other on the same circumference of the cylinder, and two slits 223 are provided at positions facing each other on the same circumference of the cylinder, but are not limited thereto. The number of the projecting portions 222 and the slits 223 may be equal to or greater than three.

In this way, the stopper 22 includes the second flange 221 on the proximal end portion. The second flange 221 is in contact with the proximal end of the grip 14. The projecting portions 222 are provided on an inner peripheral surface of the cylindrical portion 225 of the stopper 22. When the outer shaft 18 is inserted to a predetermined position, the projecting portions 222 engage with a recessed portion 213 (described below) provided on a cylindrical surface of the outer shaft 18.

In this way, the stopper 22 has, at positions adjacent to the projecting portions 222, the slits 223 from a side on which the guide sleeve 21 is inserted toward the second flange 221. A distance from a tip of each of the projecting portions 222 to an axis line of a cylindrical portion 212 of the guide sleeve 21 is smaller than a radius of the cylindrical portion 212 of the guide sleeve 21.

In this way, the cylindrical portion 225 of the stopper 22 is pushed and spread outward by providing the slits 223. Accordingly, a force such that the projecting portions 222 move in a central direction of the guide sleeve 21 acts until the projecting portions 222 of the stopper 22 engage with the recessed portion 213 of the guide sleeve 21. When the projecting portions 222 of the stopper 22 reach a position of the recessed portion 213 of the guide sleeve 21, the projecting portions 222 automatically engage with the recessed portion 213 by the force described above. This allows the projecting portions 222 of the stopper 22 and the recessed portion 213 of the guide sleeve 21 to easily engage with each other.

The slits 223 are bent toward the projecting portions 222 near the second flange 221 in the circumferential direction, and extend in the circumferential direction of the cylindrical portion 225 of the stopper 22.

The stopper 22 easily moves outward in a radius direction since the slits 223 are provided in the circumferential direction. In other words, the stopper 22 is easily rotated. When the projecting portions 222 of the stopper 22 engage with the recessed portion 213 of the guide sleeve 21, the stopper 22 can be easily rotated in only one direction by providing the slits 223 in one direction of the circumferential direction. In this way, when the stopper 22 is detached from the guide sleeve 21, rotation of the stopper 22 in one direction can be made easy, and rotation in an opposite direction can also be made difficult. More specifically, when the stopper 22 is rotated from an opposite side to a bent portion 224 of the slits 223 provided in the circumferential direction toward the bent portion 224 side, the slits 223 provided in the axial direction enable the cylindrical portion 225 to easily spread to an outer peripheral surface side. Accordingly, the stopper 22 can be easily rotated. On the other hand, when the stopper 22 is rotated from the bent portion 224 side of the slits 223 provided in the circumferential direction toward the opposite side to the bent portion 224, the cylindrical portion 225 hardly spreads, and thus the stopper 22 is hardly rotated.

Two slits 223 may be provided at positions facing each other in a cross section perpendicular to the axis line of the cylindrical portion 225 of the stopper 22. This allows a force to act toward the center side in a balanced manner when the cylindrical portion 225 moves outward in the radius direction.

Two projecting portions 222 may be provided at positions facing each other in the cross section perpendicular to the axis line of the cylindrical portion 225 of the stopper 22. This allows stable fixation of the stopper 22 and the guide sleeve 21 when the projecting portions 222 engage with the recessed portion 213 of the guide sleeve 21.

Details of the projecting portions 222 will be described with reference to FIG. 6. The projecting portions 222 are provided on the inner peripheral surface of the cylindrical portion 225. A line extending from each of side surfaces (2221, 2222, 2223, and 2224) of the projecting portions 222 in the central direction is not directed toward a center 250 of the cylindrical portion 225, and is directed toward a position forward away from the center 250. In other words, when a virtual straight line (not illustrated) is drawn from the center 250 toward each point on an outermost periphery side of each of the side surfaces 2221, 2222, 2223, and 2224 of the projecting portions 222 in a plan view, each point on an innermost periphery of each of the side surfaces 2221, 2222, 2223, and 2224 is not located on the virtual straight line. In this way, upon engagement of the stopper 22 with the recessed portion 213 of the guide sleeve 21 described below, when the stopper 22 is rotated with a predetermined force or more, the projecting portions 222 can be easily detached from the recessed portion 213. Note that, in the present embodiment, all of the side surfaces of the projecting portions 222 have the configuration described above, which is not necessarily required. The configuration described above may be satisfied by having only a side surface in contact with a wall surface of the recessed portion 213 when the stopper 22 is rotated.

In this way, in a line of sight parallel to the axis line of the cylinder of the guide sleeve 21, a point on an innermost periphery side of the side surface of the projecting portions 222 in contact with the recessed portion 213 in a rotation direction in which the stopper 22 rotates with respect to the guide sleeve 21 is present on a downstream side in the rotation direction from a straight line connecting the center of the cylindrical portion 212 of the guide sleeve 21 and a point on an outermost periphery side of the side surface.

Thus, an inner point of the side surface of the projecting portions 222 is first brought into contact with a side surface of the recessed portion 213 by rotation. Accordingly, a force that opens the projecting portions 222 outward acts to achieve an effect of easily releasing the engagement between the projecting portions 222 and the recessed portion 213.

Outer Shaft 18

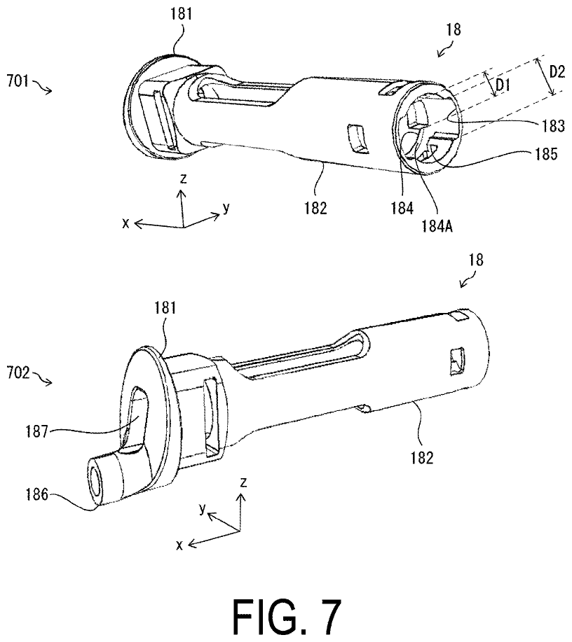

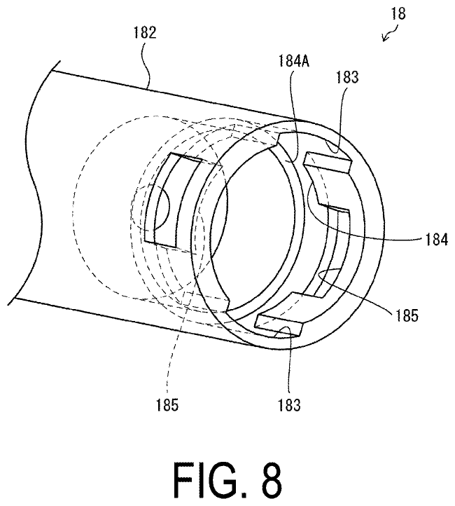

Details of the outer shaft 18 will be described with reference to FIGS. 7 and 8. FIG. 7 is a perspective view illustrating an outer appearance of the outer shaft 18. FIG. 8 is a schematic view illustrating details of a groove provided in the outer shaft 18.

As illustrated in FIG. 7, the outer shaft 18 has a cylindrical shape, and includes the first flange 181 at a distal end portion of the cylindrical portion 182. A proximal end portion of the cylindrical portion 182 on an opposite side to the first flange 181 side is open. A first groove portion 183 is provided in an inner peripheral surface of the opening portion.

A protruding portion 186 protruding outward in the axial direction is provided on an end surface on the first flange 181 side of the outer shaft 18. The second arm 13 is connected to the end surface.

A through hole 187 is provided in a cross section of the first flange 181 of the outer shaft 18. The first arm 12 connected to the shaft 15 is inserted into the through hole 187.

As illustrated in FIG. 8, the first groove portion 183 is provided at a predetermined length in parallel with the axis line of the cylinder from an end portion (first end portion) of the cylindrical portion 182 on the opposite side to the first flange 181. A second groove portion 184 is continuous with the first groove portion 183 at a position separated from the end portion and is provided in a circumferential direction of the cylindrical portion 182. The third groove portion 185 is continuous with the second groove portion 184 and is provided toward an end portion at a length not reaching the end portion.

Two first groove portions 183 are provided at positions facing each other on the same inner periphery on the inner peripheral surface of the outer shaft 18. Two third groove portions 185 are provided at positions facing each other on the same inner periphery on the inner peripheral surface of the outer shaft 18.

Guide Sleeve 21

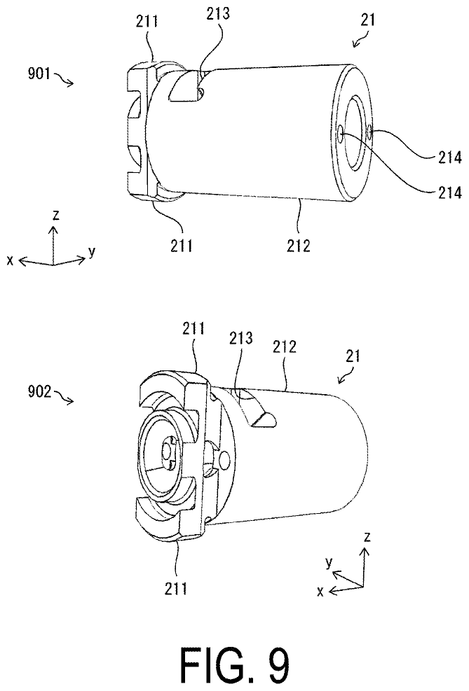

Details of the guide sleeve 21 will be described with reference to FIG. 9. FIG. 9 is a perspective view illustrating an outer appearance of the guide sleeve 21.

As illustrated in FIG. 9, the guide sleeve 21 has a cylindrical shape, and includes the protruding portions 211 on a distal portion. Two protruding portions 211 protrude in the radius direction farther than the cylindrical surface of the cylindrical portion 212, and are located at positions facing each other in the circumferential direction. The protruding portions 211 move in an order of the first groove portion 183, the second groove portion 184, and the third groove portion 185 of the outer shaft 18, and are held inside the third groove portion 185. Accordingly, the outer shaft 18 holds the guide sleeve 21.

The guide sleeve 21 is housed in an inner portion of the grip 14 with the outer shaft 18 and the stopper 22 assembled from opposite directions along the axis line of the cylinder of the guide sleeve 21.

When the projecting portions 222 of the stopper 22 engage with the recessed portion 213 provided in the cylindrical surface of the guide sleeve 21, each of the two protruding portions 211 of the stopper 22 is not movable to the second groove portion 184.

Two protruding portions 211 may be provided at positions facing each other in the cross section perpendicular to the axis line of the cylindrical portion 212 of the guide sleeve 21. Two first groove portions 183 may be provided at positions facing each other in the cross section perpendicular to the axis line of the cylindrical portion 182 of the outer shaft 18. Two third groove portions 185 may be provided at positions facing each other in the cross section perpendicular to the axis line of the cylindrical portion 182 of the outer shaft 18.

In this way, the protruding portions 211 can be easily inserted into the first groove portion 183, and the guide sleeve 21 and the outer shaft 18 can also be stably fixed to each other when the protruding portions 211 are located in the third groove portion 185.

Assembly of Stopper 22 and Guide Sleeve 21

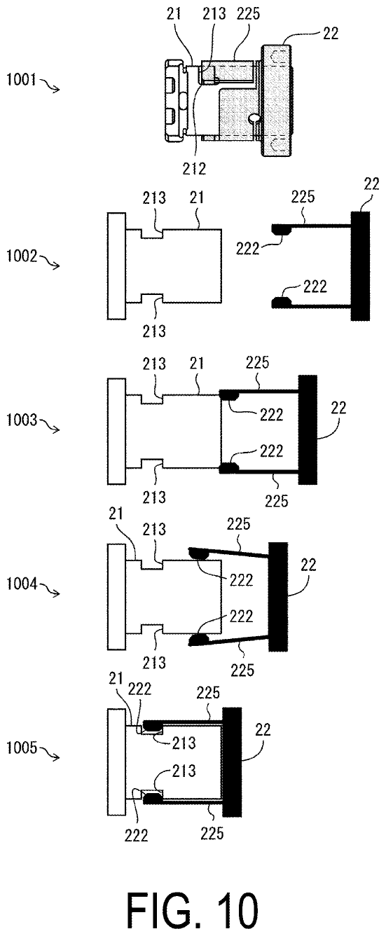

Assembly of the stopper 22 and the guide sleeve 21 will be described with reference to FIG. 10.

FIG. 10 is a diagram illustrating a mechanism for assembling the stopper 22 and the guide sleeve 21.

A reference sign 1001 in FIG. 10 illustrates a state where the stopper 22 and the guide sleeve 21 are assembled. In the state where the stopper 22 and the guide sleeve 21 are assembled, the projecting portions 222 of the stopper 22 fit into the recessed portion 213 of the guide sleeve 21 to assemble the guide sleeve 21 and the stopper 22 in a state in which detachment is less likely.

Diagrams with reference signs 1002 to 1005 illustrate more details. When the stopper 22 approaches the guide sleeve 21 as illustrated in the diagram with the reference sign 1003 from a state where the guide sleeve 21 and the stopper 22 are separated from each other as illustrated in the diagram with the reference sign 1002, the projecting portions 222 of the stopper 22 are brought into contact with the cylindrical portion 212 of the guide sleeve 21. When the stopper 22 further moves toward the guide sleeve 21, the cylindrical portion 225 of the stopper 22 is spread by an influence of the slits 223. As illustrated in the diagram with the reference sign 1004, the guide sleeve 21 enters an inner portion of the cylindrical portion 225 of the stopper 22. In this state, the cylindrical portion 225 of the stopper 22 is urged in the central direction of a cross-sectional circle of the guide sleeve 21. As illustrated in the diagram with the reference sign 1005, when the guide sleeve 21 is further pushed into the cylindrical portion 225 of the stopper 22, and the projecting portions 222 of the stopper 22 reach the position of the recessed portion 213 of the guide sleeve 21, the projecting portions 222 fit into the recessed portion 213 by an urging force. In this way, the guide sleeve 21 and the stopper 22 are assembled in a state where it is difficult to detach them from each other.

Detachment of Guide Sleeve 21 and Stopper 22

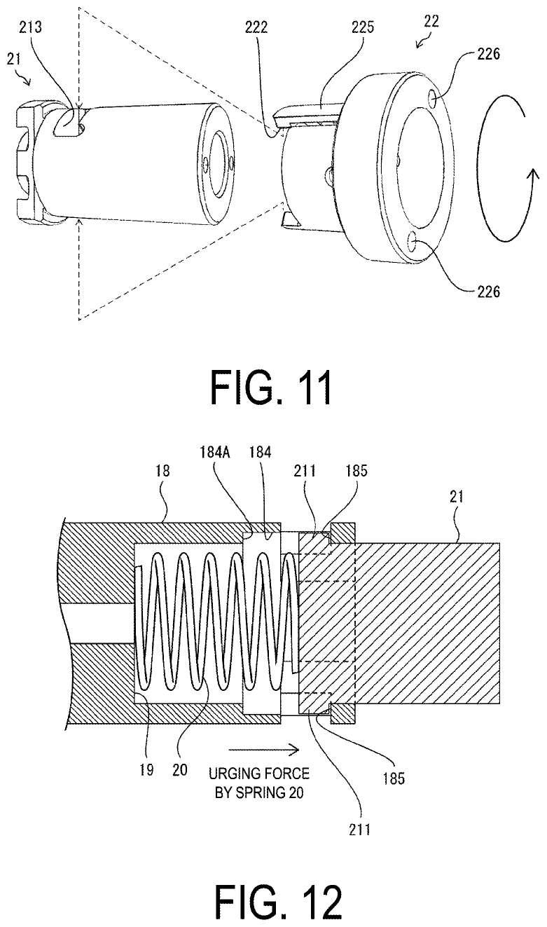

A method for detaching the stopper 22 from the guide sleeve 21 will be described with reference to FIG. 11. FIG. 11 is a diagram illustrating the method for detaching the stopper 22 from the guide sleeve 21.

As described above, when the guide sleeve 21 and the stopper 22 are assembled, the projecting portions 222 of the stopper 22 fit into the recessed portion 213 of the guide sleeve 21. In this state, when the stopper 22 is rotated in the circumferential direction with a predetermined force or more, the projecting portions 222 are detached from the recessed portion 213 due to a shape of the side surface of the projecting portions 222 of the stopper 22 being inclined toward the center. When the projecting portions 222 are detached from the recessed portion 213, and then the stopper 22 is moved in a direction away from the guide sleeve 21, the guide sleeve 21 and the stopper 22 can be detached from each other.

Note that, as described above, in order to detach the projecting portions 222 from the recessed portion 213, the stopper 22 is required to be rotated in the circumferential direction with a predetermined force or more. This can be achieved by fitting a wrench into the wrench hole 226 of the stopper 22 and rotating the stopper 22 with a predetermined force.

As described above, the engagement between the projecting portions 222 and the recessed portion 213 is released by rotating the stopper 22 in the circumferential direction of the cylindrical portion 212 of the guide sleeve 21, specifically, in a direction from the opposite side to the bent portion 224 of the slits 223 provided in the circumferential direction of the stopper 22 toward the bent portion 224 side. In this way, the stopper 22 and the guide sleeve 21 can be detached by rotating the stopper 22 in the circumferential direction of the cylindrical portion 212 of the guide sleeve 21.

Mounting of Outer Shaft 18 and Guide Sleeve 21

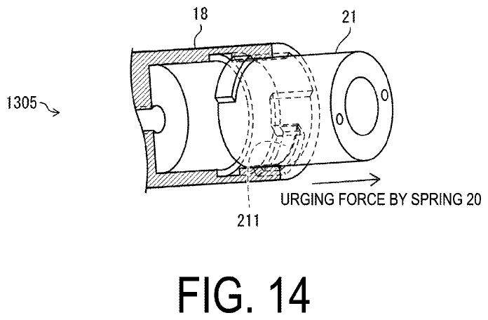

A method for mounting the guide sleeve 21 on the outer shaft 18 will be described with reference to FIGS. 12 to 14. FIGS. 12 to 14 are diagrams illustrating the method for mounting the guide sleeve 21 on the outer shaft 18.

FIG. 12 illustrates a state where the guide sleeve 21 is mounted on the outer shaft 18. As illustrated in FIG. 12, when the guide sleeve 21 is mounted on the outer shaft 18, that is, when the protruding portions 211 of the guide sleeve 21 fit into the third groove portion 185 of the outer shaft 18, the protruding portions 211 are urged by an urging force of the spring 20 in a direction in which the protruding portions 211 are not detached from the third groove portion 185. In this way, the guide sleeve 21 can be made less likely to detach from the outer shaft 18.

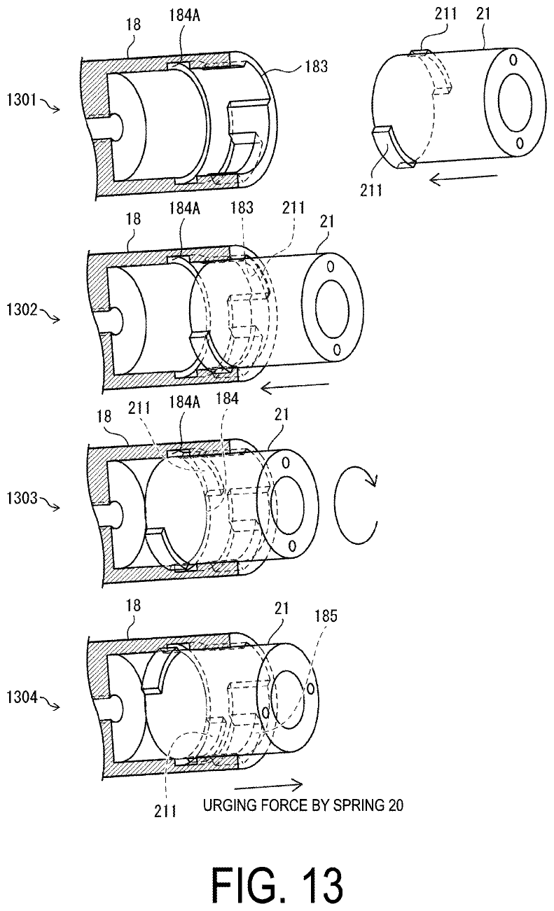

FIG. 13 illustrates a method for mounting the outer shaft 18 on the guide sleeve 21. As illustrated in a diagram with a reference sign 1301, the outer shaft 18 and the guide sleeve 21 are brought closer to each other. As illustrated in a diagram with a reference sign 1302, the guide sleeve 21 is pushed into the outer shaft 18 such that the protruding portions 211 of the guide sleeve 21 fit into the first groove portion 183 of the outer shaft 18. As illustrated in a diagram with a reference sign 1303, when the guide sleeve 21 continues to be pushed, the protruding portions 211 are brought into contact with a side surface 184A of the second groove portion 184 of the outer shaft 18.

When the protruding portions 211 are brought into contact with the side surface 184A, the guide sleeve 21 is then rotated in the circumferential direction of the outer shaft 18. As illustrated in a diagram with a reference sign 1304, when the protruding portions 211 reach a position of the third groove portion 185, the protruding portions 211 fit into the third groove portion 185 by an urging force of the spring 20, and the guide sleeve 21 becomes less likely to detach from the outer shaft 18 (a diagram with a reference sign 1305).

As described above, when the projecting portions 222 of the stopper 22 and the recessed portion 213 of the guide sleeve 21 engage with each other, the guide sleeve 21 cannot move with respect to the outer shaft 18, and thus fixation is not released. Accordingly, the medical instrument grip holder 100 that cannot be easily disassembled can be realized.

The stopper 22 and the guide sleeve 21 can be disassembled by releasing the engagement between the projecting portions 222 of the stopper 22 and the recessed portion 213 of the guide sleeve 21. The outer shaft 18 and the guide sleeve 21 can be disassembled by pushing the guide sleeve 21 to the first flange 181 side of the outer shaft 18, and moving the protruding portions 211 from the second groove portion 184 to the first groove portion 183. Since the stopper 22, the guide sleeve 21, and the outer shaft 18 can be disassembled by performing a predetermined procedure in such a manner, an instrument can be disassembled and washed.

Note that, as described above, an example in which the guide sleeve 21 is less likely to be detached from the outer shaft 18 by the urging force of the spring 20 is described, but the spring 20 is not essential. Even in the absence of the spring 20, when the projecting portions 222 of the stopper 22 engage with the recessed portion 213 of the guide sleeve 21 while the protruding portions 211 of the guide sleeve 21 are fitted into the third groove portion 185 of the outer shaft 18, the protruding portions 211 of the guide sleeve 21 cannot move to the second groove portion 184 of the outer shaft 18. The reason is that, when trying to move the protruding portions 211 of the guide sleeve 21 to the second groove portion 184 of the outer shaft 18, an end portion of the cylindrical portion 225 of the stopper 22 and an end portion of the outer shaft 18 interfere with each other and cannot be moved from the interference position.

Note that, in the presence of the spring 20, the guide sleeve 21 is biased to the opposite side to the first flange 181 inside the outer shaft 18 by the urging force of the spring 20, and thus the protruding portions 211 of the guide sleeve 21 can be easily held in the third groove portion 185.

### CONCLUSION

As described above, the medical instrument grip holder 100 according to the present embodiment includes the outer shaft 18, the guide sleeve 21, and the stopper 22 having a cylindrical shape. The stopper 22 includes, on the proximal end portion, the second flange 221 in contact with the proximal end of the grip 14, and includes, on the inner peripheral surface of the cylindrical portion 225 of the stopper 22, the projecting portions 222 configured to engage with the recessed portion 213 disposed on the cylindrical surface of the guide sleeve 21 when the guide sleeve 21 is inserted to a predetermined position. The guide sleeve 21 is housed in an inner portion of the grip 14 with the outer shaft 18 and the stopper 22 assembled from opposite directions along the axis line of the cylinder of the guide sleeve 21.

According to the configuration described above, since the projecting portions 222 of the stopper 22 and the recessed portion 213 of the guide sleeve 21 engage with each other, the projecting portions 222 and the recessed portion 213 are positioned and fixed in the engagement position. In this way, the stopper 22 and the guide sleeve 21 are fixed to each other, and the guide sleeve 21 and the outer shaft 18 are housed and fixed in the inner portion of the grip 14. Accordingly, the stopper 22 and the outer shaft 18 are fixed at a predetermined position. Thus, the medical instrument grip holder 100 that cannot be disassembled unless a predetermined procedure is performed can be realized.

The stopper 22 and the guide sleeve 21 can be disassembled by releasing the engagement between the projecting portions 222 of the stopper 22 and the recessed portion 213 of the guide sleeve 21. Since the stopper 22, the guide sleeve 21, and the outer shaft 18 can be disassembled by performing a predetermined procedure, an instrument can be disassembled and washed.

The rod inserter 1 according to the present embodiment includes the medical instrument grip holder 100, the grip 14, the first arm 12, the second arm 13, the shaft 15, and the knob 17. The second arm 13 includes the proximal end portion connected to the first flange 181 of the outer shaft 18, and holds a rod at the distal end portion. The shaft 15 is disposed in the cylinder of the cylindrical portion 182 of the outer shaft 18. The first arm 12 includes the proximal end portion connected to the shaft 15, and the rod can be attached and detached by moving the distal end portion. The knob 17 is connected to the shaft 15, and operates movement of the distal end portion of the shaft 15.

This configuration can provide the rod inserter 1 including the medical instrument grip holder 100 that cannot be easily disassembled, but can be disassembled by performing a predetermined procedure. Thus, disassembly and washing can be achieved after surgery, and accidents that cause parts of an instrument to fall into a patient's body during surgery can also be suppressed.

In the present disclosure, the invention has been described above based on the various drawings and examples. However, the invention according to the present disclosure is not limited to each embodiment described above. That is, the embodiments of the invention according to the present disclosure can be modified in various ways within the scope illustrated in the present disclosure, and embodiments obtained by appropriately combining the technical means disclosed in different embodiments are also included in the technical scope of the invention according to the present disclosure. In other words, note that a person skilled in the art can easily make various variations or modifications based on the present disclosure. Note that these variations or modifications are included within the scope of the present disclosure.

### REFERENCE SIGNS

1 Rod inserter (spinal surgical instrument)
100 Medical instrument grip holder
11 Rod
12 First arm
13 Second arm
14 Grip
Shaft (second cylindrical member)
16 Spacer
17 Knob
18 Outer shaft (first cylindrical member)
181 First flange
182 Cylindrical portion
183 First groove portion
184 Second groove portion
185 Third groove portion
19 Spring holder
20 Spring
21 Guide sleeve (junction member)
211 Protruding portion
212 Cylindrical portion
213 Recessed portion
22 Stopper
221 Second flange
222 Projecting portion
223 Slit
224 Bent portion
225 Cylindrical portion
226 Wrench hole

1. A medical instrument grip holder comprising:
a first cylindrical member;
a junction member; and
a stopper having a cylindrical shape, wherein
the stopper comprises, on a proximal end portion, a flange in contact with a proximal end of a grip and comprises, on an inner peripheral surface of a cylindrical portion of the stopper, a projecting portion configured to engage with a recessed portion disposed on a cylindrical surface of the junction member when the junction member is inserted to a predetermined position, and
the junction member is housed in an inner portion of the grip with the first cylindrical member and the stopper assembled from opposite directions along an axis line of a cylindrical portion of the junction member.

2. The medical instrument grip holder according to claim 1, further comprising:
a first groove portion, in an inner peripheral surface of a cylindrical portion of the first cylindrical member, at a predetermined length parallel to the axis line from an end portion of the cylindrical portion;
a second groove portion continuous with the first groove portion at a position separated from the end portion and provided in a circumferential direction of the cylindrical portion; and
a third groove portion continuous with the second groove portion and provided toward the end portion at a length not reaching the end portion, wherein
the junction member comprises a protruding portion protruding in a radius direction farther than the cylindrical surface of a cylindrical portion of the junction member,
the protruding portion is movable in an order of the first groove portion, the second groove portion, and the third groove portion and is held inside the third groove portion, and
the protruding portion is not movable to the second groove portion when the projecting portion of the stopper engages with the recessed portion disposed in the cylindrical surface of the junction member.

3. The medical instrument grip holder according to claim 1, wherein
the stopper comprises, at a position adjacent to the projecting portion, a slit from a side on which the junction member is inserted toward the flange, and
a distance from a tip of the projecting portion to the axis line is smaller than a radius of the cylindrical portion of the junction member.

4. The medical instrument grip holder according to claim 3, wherein
the slit is bent toward the projecting portion side near the flange in the circumferential direction and extends in the circumferential direction of the cylindrical portion of the stopper.

5. The medical instrument grip holder according to claim 3, wherein
the slit is provided as two slits disposed at positions facing each other in a cross section perpendicular to an axis line of the cylindrical portion of the stopper.

6. The medical instrument grip holder according to claim 1, wherein
the projecting portion is provided as two projecting portions disposed at positions facing each other in the cross section perpendicular to an axis line of the cylindrical portion of the stopper.

7. The medical instrument grip holder according to claim 2, wherein
the protruding portion is provided as two protruding portions disposed at positions facing each other in a cross section perpendicular to the axis line of the cylindrical portion of the junction member,
the first groove portion is provided as two first groove portions disposed at positions facing each other in a cross section perpendicular to an axis line of the cylindrical portion of the first cylindrical member, and
the third groove portion is provided as two third groove portions disposed at positions facing each other in the cross section perpendicular to the axis line of the cylindrical portion of the first cylindrical member.

8. The medical instrument grip holder according to claim 1, wherein
the projecting portion is configured to disengage with the recessed portion by rotating the stopper in the circumferential direction of the cylindrical portion of the junction member.

9. The medical instrument grip holder according to claim 8, wherein,
in a line of sight parallel to the axis line, a point on an innermost periphery side of a side surface of the projecting portion in contact with the recessed portion in a rotation direction in which the stopper rotates with respect to the junction member is present on a downstream side in the rotation direction from a straight line connecting the center of the cylindrical portion of the junction member and a point on an outermost periphery side of the side surface.

10. The medical instrument grip holder according to claim 1, wherein
the first cylindrical member comprises a flange on a distal portion, and
the flange of the first cylindrical member and the flange of the stopper sandwich the grip.

11. The medical instrument grip holder according to claim 1, wherein
the first cylindrical member, the junction member, the stopper, and the grip are configured to be repeatedly assembled and disassembled.

12. The medical instrument grip holder according to claim 1, further comprising
an urging member disposed in a cylinder of the first cylindrical member and configured to bias the junction member in a direction of the stopper.

13. The medical instrument grip holder according to claim 12, wherein
the urging member comprises any one of a coil spring, a plate spring, and an elastic polymer, or a combination thereof.

14. A spinal surgical instrument comprising:
the medical instrument grip holder according to claim 1;
a grip;
a second arm comprising a proximal end portion configured to connect to the first cylindrical member, the second arm configured to hold a rod at a distal end portion;
a second cylindrical member disposed in the cylinder of the first cylindrical member;
a first arm comprising a proximal end portion configured to connect to the second cylindrical member, the first arm configured to attach and detach the rod by moving a distal end portion; and
a knob configured to connect to the second cylindrical member, and configured to operate movement of the distal end portion of the first arm.

15. The spinal surgical instrument according to claim 14, wherein
the distal end portion of the first arm moves by rotating the knob about the axis of the first cylindrical member.
